(19) United States
(12) Patent Application Publication
(10) Pub. No.: US 2023/0105912 A1
(43) Pub. Date: Apr. 6, 2023

(54) CUSTOMIZED SKIN CARE METHOD AND SYSTEM

(71) Applicant: GPOWER INC., Seoul (KR)

(72) Inventors: Chang Hee HAN, Gyeonggi-do (KR); Deug Ki LEE, Seoul (KR)

(21) Appl. No.: 17/695,007

(22) Filed: Mar. 15, 2022

(30) Foreign Application Priority Data
Oct. 6, 2021 (KR) 10-2021-0132545

(51) Int. Cl. G16H 20/10; G16H 30/20

(52) U.S. Cl. CPC G16H 20/10; G16H 30/20

(57) ABSTRACT

A user-customized skin care method according to an embodiment of the present disclosure is capable of facilitating care for skin improvement by providing a user with customized skin care instructions through an analysis of a user skin condition by estimation models. In the customized skin care method, operation of a skin care device of a user is controlled according to skin care instructions estimated on the basis of personal information and skin condition information of the user, so that the user is provided with optimum customized skin care.

### CROSS-REFERENCE TO RELATED APPLICATION AND CLAIM OF PRIORITY

The present application claims priority to Korean Patent Application No. 10-2021-0132545, filed Oct. 6, 2021, the entire contents of which is incorporated herein for all purposes by this reference.

### BACKGROUND

### 1. Field

The present disclosure relates to a customized skin care method. In particular, the present disclosure relates to a customized skin care method and a customized skin care system, the method and the system providing users with customized skin care instructions through analysis of skin conditions of the users through estimation models, thereby facilitating care for skin improvement by the users.

### 2. Description of the Related Art

As people's interest in beauty has increased, interest in skin beauty has also increased. According to this trend, skin care apparatuses capable of measuring and analyzing skin conditions of persons and performing a variety of skin care programs accordingly have been developed and commercialized.

The conventional skin care apparatuses perform a particular skin care function on persons, specifically, people who receive skin care, according to the operation by experts, for example, doctors or skin care specialists. For example, among the conventional skin care apparatuses, a high-frequency skin care apparatus is a skin care apparatus that is used to improve skin elasticity by applying an electric current having a frequency of about 1 MHz to skin and thus stimulating the collagen of the dermis of the skin.

This high-frequency skin care apparatus is used on a person who receives skin care with appropriate intensity and time periods depending on the skin condition of the person, thereby preventing side effects from occurring on the skin of the person. Accordingly, in the related art, experts, such as, doctors or skin care specialists, who work in dermatologist offices or skin care clinics diagnose skin conditions of persons who receive skin care, set high-frequency intensity and time periods appropriate to the persons when using the high-frequency skin care apparatuses accordingly, and perform skin care.

Therefore, the people who want to receive skin care visit a particular place, specifically, dermatologist offices or skin care clinics, have their skin conditions measured, and receive skin care accordingly. That is, there are temporal or spatial limitations of skin care.

Recently, the conventional skin care apparatuses that have been used only in particular places now are manufactured for home use and used by a large number of people. However, for the conventional skin care apparatus for home use, the users have to set the apparatus operation manually, for example, a time period or intensity.

Therefore, in the related art, there is a problem that because of wrong operation setting of the skin care apparatuses by general users having no professional knowledge, skin care effects do not appear or side effects occur.

The foregoing is intended merely to aid in the understanding of the background of the present disclosure, and is not intended to mean that the present disclosure falls within the purview of the related art that is already known to those skilled in the art.

### SUMMARY

The present disclosure is directed to providing a customized skin care method and a customized skin care system, the method and the system providing users with customized skin care instructions through analysis of skin conditions of the users through estimation models, thereby facilitating care for skin improvement by the users.

According to an embodiment of the present disclosure, there is provided a customized skin care method including: generating, by a first user terminal of a first user, user information and transmitting the user information to a skin care server, the user information containing a result of measuring a skin condition of the first user transmitted from a first skin care device; estimating, by the skin care server, one or more skin care instructions based on the user information through an estimation model, generating skin care information, and transmitting the skin care information to the first user terminal, the skin care information containing the estimated skin care instructions; and controlling, by the first user terminal, operation of the first skin care device on the basis of the skin care information, and performing skin care for improving skin of the first user through the first skin care device.

According to an embodiment of the present disclosure, there is provided a customized skin care system including: a skin care device configured to measure and output a skin condition of a user; a user terminal configured to generate user information and output the user information, the user information containing a result of measuring the skin condition transmitted from the skin care device; and a skin care server configured to estimate one or more skin care instructions on the basis of the user information transmitted from the user terminal, and generate skin care information and transmit the skin care information to the user terminal, the skin care information containing the estimated skin care instructions.

Herein, the user terminal may be configured to generate an operation control signal for controlling operation of the skin care device on the basis of the skin care information, and output the operation control signal, and the skin care device may be configured to perform skin care for improving skin of the user as the operation of the skin care device is controlled according to the operation control signal.

The customized skin care method according to the present disclosure can estimate optimum skin care instructions and effects for skin care of a user according to personal information of the user, such as age, gender, or menstrual cycle, and skin condition information of the user measured through the skin care device, and can generate skin care information containing the skin care instructions accordingly, thereby controlling the user skin care operation of the skin care device.

According to the present disclosure, user-customized skin care is achieved through the skin care device that the user has, so that the convenience of skin care of the user who is a non-expert can be increased and skin improvement effects can thus be increased.

In addition, according to the customized skin care method of the present disclosure, previous skin care instructions and effects are re-estimated according to skin condition information remeasured through the skin care device after user skin care is performed, so that new skin care information can be generated.

Thus, the present disclosure can advance the estimation of instructions and effects by the estimation models according to actual skin improvement effects, and can increase the accuracy of estimation, thereby providing the user with optimum customized skin care instructions.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a diagram illustrating a configuration of a customized skin care system according to an embodiment of the present disclosure;

FIG. 2 is a diagram illustrating a configuration of a skin care device of FIG. 1;

FIG. 3 is a diagram illustrating a configuration of a user terminal of FIG. 1;

FIG. 4 is a diagram illustrating a configuration of a skin care server of FIG. 1;

FIG. 5 is a flowchart illustrating a customized skin care method according to an embodiment of the present disclosure; and

FIG. 6 is a flowchart illustrating a customized skin care method according to another embodiment of the present disclosure.

### DETAILED DESCRIPTION

The terms used in the present specification and the claims are general terms selected in consideration of function of corresponding elements used in various embodiments of the present disclosure. However, the terms may vary depending on intention of those skilled in the related art, legal or technical interpretation, emergence of new technologies, and the like. Further, some of the terms may be ones arbitrarily selected by the applicant. These terms may be interpreted as having the meanings defined herein. If the terms are not specifically defined, the terms may be interpreted on the basis of the general content of the present disclosure and common technical knowledge in the related art.

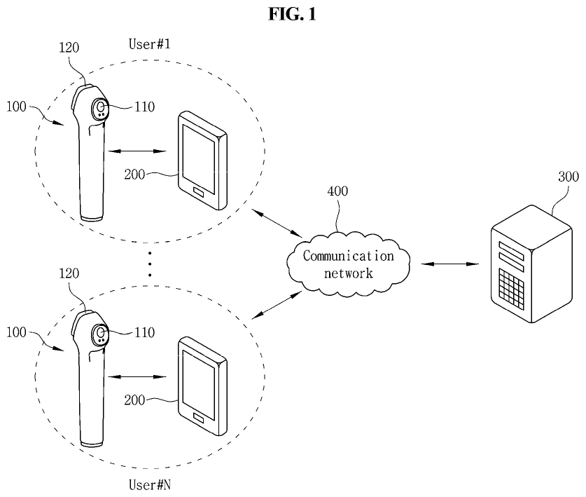

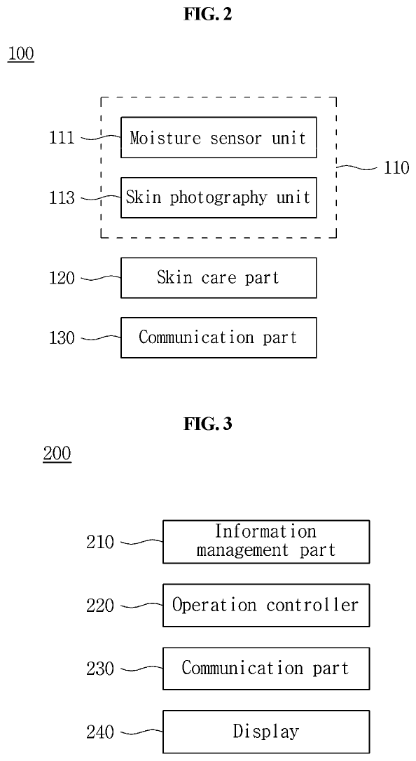

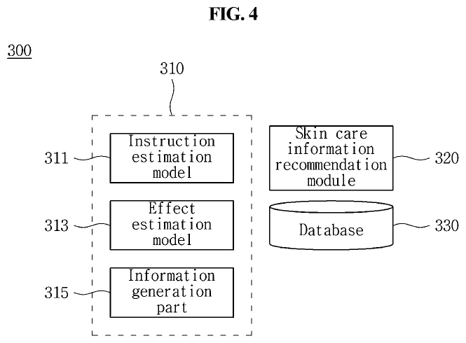

FIG. 1 is a diagram illustrating a configuration of a customized skin care system according to an embodiment of the present disclosure. FIG. 2 is a diagram illustrating a configuration of a skin care device of FIG. 1. FIG. 3 is a diagram illustrating a configuration of a user terminal of FIG. 1. FIG. 4 is a diagram illustrating a configuration of a skin care server of FIG. 1.

Referring to the figures, a customized skin care system according to an embodiment may include a skin care server 300. The skin care server 300 transmits a customized skin care instruction to each of a plurality of users, and controls the operation of a skin care device 100 of each user with the instruction so as to improve the skin of each user.

Each of the users may have the skin care device 100 and a user terminal 200 connected thereto.

The skin care device 100 may be connected to the user terminal 200 through wireless communication, for example, a communication method such as Bluetooth or Wi-Fi. The skin care device 100 may measure the user skin condition, transmit data thereof to the user terminal 200, and perform a skin care operation for improving the user skin under the control of the user terminal 200.

As shown in FIG. 2, the skin care device 100 may include a skin measurement part 110, a skin care part 120, and a communication part 130.

The skin measurement part 110 may include a moisture sensor unit 111 and a skin photography unit 113. The moisture sensor unit 111 detects the user skin condition, for example, a skin moisture state. The skin photography unit 113 photographs the user skin by emitting predetermined light to the user skin.

The moisture sensor unit 111 may include a first moisture sensor (not shown) and a second moisture sensor (not shown). The first moisture sensor detects the user skin moisture content. The second moisture sensor detects the user skin moisture loss, that is, transepidermal water loss (TEWL).

The moisture sensor unit 111 may detect either the moisture content or the transepidermal water loss of the user skin or both, and may output skin moisture information, that is, a skin barrier index of the user.

The skin photography unit 113 may include a plurality of optical elements (not shown) and a photography part. Each of the plurality of optical elements emits white light or ultraviolet light to the user skin. The photography part photographs the skin to which light has been emitted.

In addition, the skin photography unit 113 may further include a polarizing filter (not shown) that is moved by a controller (not shown) so as overlap a light emission surface of at least one of the plurality of optical elements. Thus, the optical element overlapped by the polarizing filter may emit polarized light to the user skin.

The skin photography unit 113 may use the optical elements and the polarizing filter to emit at least one of the following to the user skin: white light, ultraviolet light, and polarized light. Next, the skin photography unit 113 may output a skin image by photographing the skin to which light has been emitted, through the photography part. The skin image may contain the amount of sebum secretion, the degree of wrinkle formation, a colored area of melanic pigment, or the size of skin pores of the user.

Accordingly, the skin measurement part 110 according to the embodiment may generate skin condition information of the user from the skin moisture information output from the moisture sensor unit 111 and the skin image output from the skin photography unit 113. The skin condition information may be transmitted to the user terminal 200 of the user through the communication part 130.

The skin care part 120 may generate, on the basis of the operation control signal transmitted from the user terminal 200, the skin care operation for improving the user skin. The skin care part 120 may be at least one of the following units: a high-frequency/micro-current unit (not shown) for applying a high-frequency or a micro-current to the user skin; an iontophoresis unit (not shown) for applying ions, such as vitamin C ions, to the user skin; a light therapy unit (not shown) for emitting light of a predetermined wavelength to the user skin; and a vibration/roller unit (not shown) for applying vibration or pressure to the user skin.

In the meantime, the skin measurement part 110 and the skin care part 120 of the skin care device 100 described above may be provided in opposite directions from each other at respective ends of the skin care device 100, for example, end parts that come into contact with the user skin. Thus, while holding a handle (not shown) of the skin care device 100, the user may turn the skin care device 100 to bring the skin measurement part 110 or the skin care part 120 into contact with the user skin.

Herein, the skin measurement part 110 and the skin care part 120 may come into contact with the user skin, for example, face, arms, legs, and trunk.

The communication part 130 may transmit the skin condition information of the user output from the skin measurement part 110, to the user terminal 200. In addition, the communication part 130 may provide the skin care part 120 with the operation control signal transmitted from the user terminal 200 so that the user skin care operation of the skin care part 120 is controlled.

The user terminal 200 may be connected to the skin care server 300 over a communication network 400. The user terminal 200 may generate user information from personal information collected from the user and the skin condition information transmitted from the skin care device 100 described above, and may transmit the user information to the skin care server 300. Herein, the personal information may contain information on the user gender, age, menstrual cycle, or whether the user has a disease.

In addition, the user terminal 200 may control the operation of the skin care device 100 on the basis of skin care information of skin care data transmitted from the skin care server 300, for example, skin care instruction information generated only for the user.

The user terminal 200 may be a device, such as a computer, a laptop PC, a tablet PC, or a smartphone, capable of communicating with the skin care server 300.

Referring to FIG. 3, the user terminal 200 may include an information management part 210, an operation controller 220, a communication part 230, and a display 240.

The information management part 210 may generate user information from the personal information of the user and the skin condition information transmitted from the skin care device 100. In addition, the information management part 210 may store therein the skin care data of the user which is transmitted from the skin care server 300 corresponding to the user information. Each time the user terminal 200 accesses the skin care server 300, the information management part 210 may generate the skin care data in order and store the same therein.

Herein, the skin care data may contain user information, and skin care information that is based on instruction information and effect information generated by the skin care server 300 on the basis of the user information.

In the meantime, the information management part 210 may provide, through the display 240, the user with a questionnaire composed of one or more questions, may receive answers to the questions of the questionnaire from the user, and may store the answers as questionnaire information. The questionnaire information may be contained in the above-described user information and stored.

Herein, the questionnaire information may contain information on a skin care part that the user wants and information on a skin care effect.

The operation controller 220 may generate, on the basis of the skin care instruction information of the skin care data transmitted from the skin care server 300, an operation control signal for controlling the operation of the skin care device 100. The operation controller 220 may transmit the operation control signal to the skin care part 120 of the skin care device 100.

Herein, the operation control signal may be a signal for controlling the operation time period or the operation intensity of the skin care part 120. In addition, in the case in which the skin care part 120 is composed of a plurality of management units, operation control signals may be signals for controlling an operation sequence of each of the plurality of management units and an operation time period and an operation intensity of each of the management units.

In the meantime, the operation controller 220 may display, through the display 240, an explanation screen for controlling the operation of the skin care device 100 according to the operation control signal. Thus, referring to the explanation screen displayed on the display 240, the user may perform skin care using the skin care device 100.

The communication part 230 may receive skin condition information from the skin care device 100 and may provide the same to the information management part 210. In addition, the communication part 230 may transmit user information generated from the information management part 210 to the skin care server 300, and may receive skin care data transmitted corresponding to the user information from the skin care server 300 and provide the skin care data to the information management part 210.

The display 240 may display various screens for the user skin care to the user, for example, an information input screen for collecting personal information or questionnaire information of the user, an information display screen for displaying the skin care data of the user transmitted from the skin care server 300, or an explanation screen for controlling the operation of the skin care device 100 according to an operation control signal.

The skin care server 300 may estimate, on the basis of the user information transmitted from the user terminal 200, an instruction for user skin care and an effect thereof through one or more estimation models.

The skin care server 300 may generate skin care information based on the skin care instruction information and the skin care effect information according to a result of prediction together with the user information transmitted from the user terminal 200 of the user.

The skin care server 300 may transmit the skin care information to the user terminal 200 of the user or to a user terminal 200 of another user.

The skin care server 300 may include a skin care information generation module 310, a skin care information recommendation module 320, and a database 330.

The skin care information generation module 310 may include an instruction estimation model 311, an effect estimation model 313, and an information generation part 315.

The instruction estimation model 311 may estimate, on the basis of the user information transmitted from the user terminal 200, one or more skin care instructions appropriate for the user, and may output the same.

For example, the instruction estimation model 311 may estimate the skin care instructions on the basis of the personal information of the user information, specifically, the user age, gender, or menstrual cycle. In addition, the instruction estimation model 311 may estimate the skin care instructions on the basis of the skin condition information of the user information. The instruction estimation model 311 may estimate the skin care instructions of the user by synthesizing the personal information and the skin condition information.

The instruction estimation model 311 may be an estimation model of a machine learning method, such as an artificial neural network (ANN), a deep neural network (DNN), a convolutional neural network (CNN), or a regression analysis.

Thus, the instruction estimation model 311 performs training multiple times using skin care information for each user stored in the database 330 as training data. Accordingly, the instruction estimation model 311 may estimate one or more skin care instructions from the user information transmitted from the user terminal 200 and may output the same.

In addition, the instruction estimation model 311 may improve the estimation model on the basis of skin condition improvement information transmitted from the user terminal 200, for example, performing retraining using the skin condition improvement information. Through such improvement, the instruction estimation model 311 may re-estimate previous skin care instructions on the basis of the skin condition improvement information. The re-estimated skin care instructions may be generated as new skin care information by the information generation part 315, which will be described later.

Herein, the skin condition improvement information may be skin condition information that is generated by remeasuring the user skin condition through the skin care device 100 after the user skin care is performed through the skin care device 100 according to the skin care information of the user transmitted to the user terminal 200.

In the meantime, when user information is transmitted from the user terminal 200 of a user of which skin care information is not stored in the database 330, the instruction estimation model 311 may output one of preset multiple skin care standard instructions as skin care instruction information of the user.

Herein, the skin care standard instructions may be skin care instructions for people who have the personal information, such as age or gender, the same as the personal information of the user information transmitted from the user terminal 200.

Alternatively, the skin care standard instructions may be skin care instructions for people who have the skin condition information that is the same as the skin condition information of the user information of the user.

The effect estimation model 313 may estimate, for output, one or more user skin improvement effects, that is, skin care effects, on the basis of the user information transmitted from the user terminal 200 and the skin care instructions output from the instruction estimation model 311 described above.

For example, the effect estimation model 313 may estimate skin care effects of the user from the personal information of the user information and the skin care instructions estimated on the basis thereof. In addition, the effect estimation model 313 may estimate skin care effects of the user from the skin condition information of the user information and the skin care instructions estimated on the basis thereof. The effect estimation model 313 may estimate the skin care effects of the user from the skin care instructions that are estimated by synthesizing the personal information and the skin condition information.

Herein, the skin care effects estimated by the effect estimation model 313 may include: skin moisture effect values such as an increase of a skin moisture content of the user and a reduction of transepidermal water loss; or skin condition effect values such as a reduction of the amount of sebum secretion, an increase of the amount of wrinkle improvement, and a reduction of size of skin pores.

In addition, the effect estimation model 313 may re-estimate previous skin care effects on the basis of the skin care instructions re-estimated by the instruction estimation model 311 described above. The re-estimated skin care effects may be generated as new skin care information by the information generation part 315, which will be described later.

The effect estimation model 313 may be an estimation model of the same machine learning method as the above-described instruction estimation model 311. The effect estimation model 313 may estimate one or more user skin care effects through multiple times of training using the skin care information for each user stored in the database 330 as training data, and may output the same.

The information generation part 315 may generate skin care information for each user by synthesizing the one or more skin care instructions estimated by the instruction estimation model 311, the one or more skin care effects estimated by the effect estimation model 313, and the user information transmitted from the user terminal 200.

The skin care information of the user may be stored in the database 330 and may be used as training data for the instruction estimation model 311 and the effect estimation model 313. In addition, the skin care information of the user may be transmitted to the user terminal 200 of the user.

The skin care information recommendation module 320 may extract and post one or more pieces of skin care information of respective multiple users stored in the database 330.

Thus, the multiple users may access the skin care server 300 through their user terminals 200, may find the skin care information of other users posted on the server 300, and may download the skin care information they want to their user terminals 200.

In addition, the skin care information recommendation module 320 may extract at least one of the multiple pieces of posted skin care information and may transmit the extracted information to the user terminal 200 of a particular user among the multiple users for recommendation.

Herein, the skin care information recommendation module 320 may transmit skin care effects of the particular user estimated by the above-described effect estimation model 313 together. Herein, the effect estimation model 313 may estimate the skin care effects of the particular user on the basis of the user information of the particular user stored in the database 330 and the skin care information extracted by the skin care information recommendation module 320.

The skin care information recommendation module 320 may extract skin care information of a first user among the multiple pieces of posted skin care information. Next, from the skin care information of the multiple users, the skin care information recommendation module 320 may extract a user who has the same personal information as the first user, for example, at least one of the first user's age, gender, and menstrual cycle. The skin care information recommendation module 320 may transmit the skin care information of the first user to the user terminal 200 of the extracted user.

In addition, from the skin care information of the multiple users, the skin care information recommendation module 320 may extract a user who has the same skin condition information as the first user within an error range, for example, skin moisture content, transepidermal water loss, or a skin image of skin pores or sebum conditions. Next, the skin care information recommendation module 320 may transmit the skin care information of the first user to the user terminal 200 of the extracted user.

Thus, the customized skin care system according to the embodiment enables skin care information sharing between the users who satisfy a particular condition, for example, a condition that pieces of personal information or skin condition information of the users are the same. Herein, the skin care information shared between the users may contain at least one of the following: skin condition information, skin care instructions, and skin care effects of the users.

In the database 330, skin care information for each of a plurality of users may be stored. The skin care information is arranged in order and stored each time each of the users accesses the skin care server 300 through his or her user terminal 200, in other words, each time skin care information containing skin care instructions is transmitted from the skin care server 300 to the user terminal 200.

As described above, the customized skin care system according to the embodiment may estimate optimum skin care instructions and effects for skin care of a user according to personal information of the user, such as age, gender, or menstrual cycle, and skin condition information of the user measured through the skin care device 100, and may generate skin care information containing the optimum skin care instructions accordingly. In addition, the customized skin care system controls the operation of the skin care device 100 of the user through the generated skin care information, thereby providing the user with customized skin care.

Accordingly, the customized skin care system of the present disclosure enables a user who is a non-expert to perform optimum skin care with the skin care device 100 according to his or her skin condition, thereby increasing the convenience of skin care of the user and thus increasing skin improvement effects.

In addition, the customized skin care system according to the embodiment may achieve improvement that the estimation by the instruction estimation model 311 and the effect estimation model 313 is advanced on the basis of the skin condition information, that is, skin condition improvement information, measured after the user performs skin care with the skin care device 100. Thus, the advancement of the estimation models increases the accuracy of estimation of skin care instructions and effects, so that the user is provided with optimum customized skin care instructions.

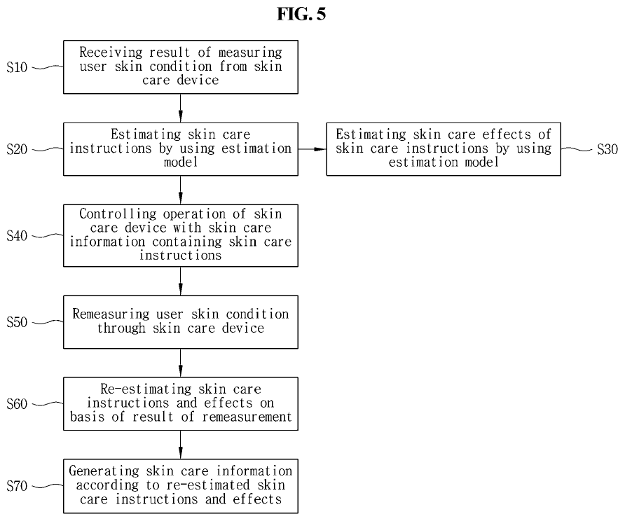

FIG. 5 is a flowchart illustrating a customized skin care method according to an embodiment of the present disclosure. Hereinafter, for convenience of description, described is a customized skin care method for one of a plurality of users, that is, a first user.

A user terminal 200 of the first user (hereinafter, referred to as a first user terminal 200) is connected to a skin care device 100 of the first user (hereinafter, referred to as a first skin care device 100, and may receive a result of measuring a skin condition of the user from the first skin care device 100, at step S10.

The first skin care device 100 may measure moisture content or transepidermal water loss of the user skin through one or more moisture sensors of a skin measurement part 110, and may photograph the skin through a photography part. The skin measurement part 110 may generate skin condition information of the user from skin moisture information of the measured skin moisture content or the measured transepidermal water loss and the skin image of the photographed skin, and may transmit the skin condition information to the first user terminal 200.

The first user terminal 200 may generate user information from personal information collected from the user and the skin condition information transmitted from the skin care device 100 described above, and may transmit the user information to a skin care server 300. Herein, the personal information may contain information on the user gender, age, menstrual cycle, or whether the user has a disease.

Next, the skin care server 300 may estimate one or more skin care instructions on the basis of the user information through an instruction estimation model 311, at step S20, and may estimate one or more skin care effects on the basis of the user information and the estimated skin care instructions through an effect estimation model 313, at step S30.

Herein, the instruction estimation model 311 may estimate the skin care instructions on the basis of the personal information of the user information, such as age, gender, or menstrual cycle of the user. In addition, the instruction estimation model 311 may estimate the skin care instructions on the basis of the skin condition information of the user information, such as the skin moisture content, the transepidermal water loss, or the skin image of the user.

For example, regarding the menstrual cycle in women of childbearing age, the following phases are repeated: the menstrual phase from the start time point of menstruation to the time point one week before ovulation; the follicular phase from the time point one week before ovulation to the ovulation phase; the ovulation phase from the ovulation phase to the time point one week before menstruation; and the luteal phase from the time point one week before menstruation to the time point immediately before menstruation. In addition, the amounts of hormone secretion of estrogen and progesterone may vary from phase to phase of the menstrual cycle.

Depending on the changes in the amounts of hormone secretion, the skin of women of childbearing age has skin condition changes, such as increase in dead skin cells or sebum, edema, or decrease in skin regeneration or immune function. Therefore, considering this, user skin care needs to be performed.

Thus, the instruction estimation model 311 according to the embodiment may estimate skin care instructions on the basis of the user menstrual cycle of the user information. For example, when the current time point, specifically, the time point at which the first user terminal 200 transmits the user information to the skin care server 300, is in the luteal phase of the user's menstrual cycle, the instruction estimation model 311 estimates one or more skin care instructions for mild-stimulation skin care to be performed through the first skin care device 100. In addition, when the current time point is in the follicular phase of the user's menstrual cycle, the instruction estimation model 311 estimates one or more skin care instructions for collagen regeneration in the user skin to be achieved through the first skin care device 100.

The skin care instructions estimated by the instruction estimation model 311 may contain information for controlling the operation of the first skin care device 100, for example, device control information for controlling the operation sequence, the operation time period, or the operation intensity of the first skin care device 100.

In addition, the skin care effects estimated by the effect estimation model 313 may include information on a skin condition improved according to the pre-estimated skin care instructions. For example, the information may include: skin moisture effect values such as an increase of a skin moisture content of the user and a reduction of transepidermal water loss; or skin condition effect values such as a reduction of the amount of sebum secretion, an increase of the amount of wrinkle improvement, and a reduction of size of skin pores.

An information generation part 315 of the skin care server 300 may generate skin care information of the user containing the estimated skin care instructions, the estimated skin care effects, and the user information, and may transmit the skin care information to the first user terminal 200. In addition, the information generation part 315 may store the generated skin care information for each user in a database 330.

Next, the first user terminal 200 may control the operation of the first skin care device 100 on the basis of the received skin care information, and the user may perform skin care for improving his or her skin condition through the first skin care device 100 of which the operation is controlled, at step S40.

Herein, the first user terminal 200 may generate an operation control signal for the first skin care device 100 on the basis of the skin care instructions of the skin care information, and may transmit the operation control signal to the first skin care device 100 to control at least one of the operation sequence, the operation time period, and the operation intensity of the first skin care device 100.

In addition, the first user terminal 200 may display, through a display 240, the operation control state of the first skin care device 100 to the first user. Thus, referring to the screen displayed on the display 240, the first user may perform skin care using the first skin care device 100.

Next, after user skin care through the first skin care device 100 is finished, the first user terminal 200 may control the operation of the first skin care device 100 to remeasure the user skin condition through the first skin care device 100, at step S50.

The first skin care device 100 may transmit skin moisture information and a skin image that are remeasured through the one or more moisture sensors and the photography part, as skin condition information to the first user terminal 200. The first user terminal 200 may generate skin condition improvement information from the results of remeasurement transmitted from the first skin care device 100, and may transmit the skin condition improvement information to the skin care server 300.

Subsequently, a skin care information generation module 310 of the skin care server 300 improves the instruction estimation model 311 on the basis of the skin condition improvement information transmitted from the first user terminal 200, and skin care instructions may be re-estimated through the improved instruction estimation model 311. Herein, the instruction estimation model 311 may be improved by performing retraining using the skin condition improvement information as training data, at step S60.

In addition to this, the effect estimation model 313 of the skin care information generation module 310 may re-estimate skin care effects on the basis of the re-estimated skin care instructions. In addition, according to another embodiment of the present disclosure, the effect estimation model 313 may generate actual skin condition improvement effects based on the skin condition improvement information, as skin care effects.

Next, the information generation part 315 of the skin care information generation module 310 may generate new skin care information by updating the pre-generated skin care information according to the re-estimated skin care instructions the re-estimated skin care effects, at step S70.

Then, the skin care server 300 may store the new skin care information in the database 330 and may transmit the same to the first user terminal 200. Thus, the first user terminal 200 may display the new skin care information to the user or may use the new skin care information to control the skin care operation of the first skin care device 100 again, thereby performing care for skin improvement again.

As described above, the skin care method according to the embodiment may estimate optimum skin care instructions and effects for skin care of a user according to personal information of the user, such as age, gender, or menstrual cycle, and skin condition information of the user measured through the skin care device 100, and may control the operation of the skin care device 100 of the user through skin care information containing the estimated skin care instructions, thereby providing the user with customized skin care.

Accordingly, the present disclosure enables a user who is a non-expert to perform optimum skin care appropriate for him or her with the skin care device 100 according to his or her skin condition, thereby increasing the convenience of skin car of the user and thus increasing skin improvement effects.

In addition, the skin care method according to the embodiment may improve the estimation models according to the skin condition information, that is, skin condition improvement information, measured after the user performs skin care with the skin care device 100, and may thus re-estimate previous skin care instructions and effects, thereby generating new skin care information.

Accordingly, the present disclosure may advance the estimation of instruction and effects by the estimation models 311 and 313 according to actual skin improvement effects, and may increase the accuracy of estimation of skin care instructions and effects, thereby providing the user with optimum customized skin care instructions.

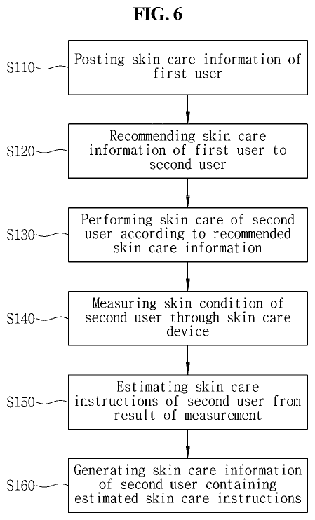

FIG. 6 is a flowchart illustrating a customized skin care method according to another embodiment of the present disclosure.

A skin care server 300 may extract skin care information of a first user (hereinafter, referred to as first skin care information) among pieces of skin care information of respective multiple users stored in a database 330, and may post the skin care information of the first user at step S110.

Herein, the first skin care information may contain user information of the first user, skin care instructions, and skin care effects corresponding thereto.

Thus, the multiple users except the first user may access the skin care server 300 through their user terminal 200, and may download the posted first skin care information optionally. Then, each user may control the operation of his or her skin care device 100 by using the downloaded first skin care information, thereby performing care for skin improvement through the skin care device 100.

Herein, the skin care server 300 may determine the order of pieces of skin care information of the respective multiple users, and may post the pieces of skin care information in order. Herein, the skin care server 300 may determine the order of pieces of the skin care instructions of the skin care information or the order of pieces of the user information of the skin care information, and may post the pieces of skin care information in order.

In addition, from the pieces of skin care information of the respective multiple users stored in the database 330, the skin care server 300 may search for and extract another user, for example, a second user, corresponding to the first skin care information.

From the pieces of skin care information stored in the database 330, a skin care information recommendation module 320 of the skin care server 300 may search for and extract another user, that is, the second user, who has the personal information that is the same as the personal information of the user information of the first skin care information, for example, at least one of the first user's age, gender, and menstrual cycle.

In addition, from the multiple pieces of skin care information, the skin care information recommendation module 320 may search for and extract a second user who has the skin condition information that is the same as the skin condition information of the user information of the first skin care information, for example, at least one of the first user's skin moisture information and skin image.

Next, the skin care server 300 may transmit the first skin care information to the user terminal 200 of the extracted second user, and may recommend the first skin care information at step S120.

Herein, the skin care server 300 may estimate skin care effects of the second user based on the first skin care information through an effect estimation model 313, and may transmit the estimated skin care effects together with the first skin care information to the user terminal 200 of the second user.

Thus, through the first skin care information and skin care effects corresponding thereto displayed on the user terminal 200 of the second user, the second user may determine whether to use the first skin care information, that is, whether to use the skin care instructions of the first user.

The user terminal 200 of the second user may control the operation of the skin care device 100 of the second user according to the received first skin care information, so that skin care for improving the skin condition of the second user is performed at step S130.

As described above, the user terminal 200 of the second user may generate, from the first skin care information, an operation control signal for controlling the operation sequence, the operation time period, or the operation intensity of the skin care device 100, and may transmit the operation control signal to the skin care device 100 so as to control the operation.

Then, when the skin care of the second user through the skin care device 100 is finished, the skin care device 100 of the second user measures the skin condition of the second user. The skin care device 100 of the second user may transmit information on the measured skin condition, that is, skin condition information of the second user, to the skin care server 300 via the user terminal 200 of the second user at step S140.

Next, on the basis of the user information of the second user stored in the database 330 and the skin condition information transmitted from the user terminal 200 of the second user, the skin care server 300 may estimate skin care instructions of the second user through an instruction estimation model 311 at step S150.

In addition, the effect estimation model 313 of the skin care server 300 may estimate skin care effects on the basis of the estimated skin care instructions of the second user.

Next, an information generation part 315 may generate skin care information of the second user by synthesizing the estimated skin care instructions and the estimated effects of the second user and the user information of the second user at step S160.

As described above, the skin care method according to the embodiment enables recommendation for and sharing of skin care information between users who, among a plurality of users, satisfy a particular condition, for example, a condition that the users have the same personal information or skin condition information or both. Accordingly, the present disclosure can increase the convenience of skin care of a user and thus increase skin improvement effects.

Although embodiments of the present disclosure have been described, those skilled in the art will appreciate that without departing from the idea of the present disclosure as disclosed in the claims, various modifications and variations of the present disclosure can be made by addition, replacement, or deletion of elements, and are included in the scope of the present disclosure.

Further, the terms described above are defined in consideration of the functions in the present disclosure, which may vary depending on the intention of a user or operator, or the custom. Therefore, the definitions of the terms should be based on the content throughout the specification.

What is claimed is:

1. A customized skin care method, comprising:
generating, by a first user terminal of a first user, user information and transmitting the user information to a skin care server, the user information containing a result of measuring a skin condition of the first user transmitted from a first skin care device;
estimating, by the skin care server, one or more skin care instructions based on the user information through an estimation model, generating skin care information, and transmitting the skin care information to the first user terminal, the skin care information containing the estimated skin care instructions; and
controlling, by the first user terminal, operation of the first skin care device on the basis of the skin care information, and performing skin care for improving skin of the first user through the first skin care device.

2. The customized skin care method of claim 1, wherein the user information contains personal information that is at least one selected from a group of an age, a gender, and a menstrual cycle of the first user; and
the generating of the skin care information and the transmitting of the skin care information to the first user terminal comprises:
estimating the one or more skin care instructions based on the personal information by using an instruction estimation model;
estimating one or more skin care effects based on the personal information and the skin care instructions by using an effect estimation model; and
generating the skin care information by combining the user information, the skin care instructions, and the skin care effects.

3. The customized skin care method of claim 1, wherein the user information contains skin condition information that is at least one selected from a group of skin moisture content, transepidermal water loss, and a skin image of the first user; and
the generating of the skin care information and the transmitting of the skin care information to the first user terminal comprises:
estimating the one or more skin care instructions based on the skin condition information by using an instruction estimation model;
estimating one or more skin care effects based on the skin condition information and the skin care instructions by using an effect estimation model; and
generating the skin care information by combining the user information, the skin care instructions, and the skin care effects.

4. The customized skin care method of claim 1, further comprising, after the performing of the skin care for improving the skin of the first user through the first skin care device:
remeasuring the skin condition of the first user by the first skin care device, and generating, by the first user terminal, skin condition improvement information from a result of remeasurement and transmitting the skin condition improvement information to the skin care server;
improving, by the skin care server, the estimation model on the basis of the skin condition improvement information, and re-estimating the skin care instructions through the improved estimation model; and
updating the skin care information on the basis of the re-estimated skin care instructions.

5. The customized skin care method of claim 1, wherein the performing of the skin care for improving the skin of the first user through the first skin care device comprises:
generating, by the user terminal, an operation control signal for controlling the operation of the first skin care device on the basis of the skin care information, and transmitting the operation control signal to the first skin care device to control the operation of the first skin care device,
wherein the operation control signal is a signal for controlling at least one selected from a group of an operation sequence, an operation time period, and an operation intensity of the first skin care device.

6. The customized skin care method of claim 1, further comprising:
searching for and extracting, by the skin care server, a second user that corresponds to the skin care information of the first user among pieces of the skin care information of respective multiple users stored in a database;
transmitting, by the skin care server, the skin care information of the first user to a user terminal of the second user;
controlling, by the user terminal of the second user, operation of a skin care device of the second user on the basis of the skin care information of the first user, and performing skin care of the second user; and
measuring a skin condition of the second user by the skin care device of the second user, and transmitting, to the skin care server by the user terminal of the second user, skin condition information based on a result of measuring the skin condition transmitted from the skin care device of the second user.

7. The customized skin care method of claim 6, wherein the transmitting of the skin care information of the first user comprises:
estimating, by the skin care server, skin care effects of the second user on the basis of the skin care information of the first user; and
transmitting the estimated skin care effects and the skin care information of the first user, to the user terminal of the second user.

8. A customized skin care system, comprising:
a skin care device configured to measure and output a skin condition of a user;
a user terminal configured to generate user information and output the user information, the user information containing a result of measuring the skin condition transmitted from the skin care device; and
a skin care server configured to estimate one or more skin care instructions on the basis of the user information transmitted from the user terminal, and generate skin care information and transmit the skin care information to the user terminal, the skin care information containing the estimated skin care instructions,
wherein the user terminal is configured to generate an operation control signal for controlling operation of the skin care device on the basis of the skin care information, and output the operation control signal, and the skin care device is configured to perform skin care for improving skin of the user as the operation of the skin care device is controlled according to the operation control signal.

9. The customized skin care system of claim 8, wherein the skin care device comprises:
a skin measurement part configured to measure the skin condition of the user; and
a skin care part composed of one or more skin care units, and configured to perform the skin care by controlling, on the basis of the operation control signal transmitted from the user terminal, at least one selected from a group of an operation sequence, an operation time period, and an operation intensity of the skin care units.

10. The customized skin care system of claim 8, wherein the skin care server comprises:
an instruction estimation model configured to estimate the skin care instructions on the basis of personal information or skin condition information of the user information or both;
an effect estimation model configured to estimate one or more skin care effects on the basis of the user information and the skin care instructions; and
an information generation part configured to generate the skin care information by combining the user information, the skin care instructions, and the skin care effects.

11. The customized skin care system of claim 10, wherein the skin care server is configured to:
receive, from the user terminal, skin condition improvement information on the skin condition of the user remeasured through the skin care device; and
improve the instruction estimation model on the basis of the skin condition improvement information and re-estimate the skin care instructions.

12. The customized skin care system of claim 8, wherein the skin care server further comprises:
a skin care information recommendation module configured to extract and post the skin care information of at least one user among pieces of the skin care information of respective multiple users stored in a database, and extract another user corresponding to the posted skin care information to recommend the posted skin care information.
